(19) United States
(12) Patent Application Publication
(10) Pub. No.: US 2007/0053923 A1
(43) Pub. Date: Mar. 8, 2007

(54) Dna vaccine composition with enhanced immunogenicity

(76) Inventors: Ki-Seok Park, Ulsan (KR); Su-Hyung Park, Seoul (KR); Se-Hwan Yang, Gyeongsangbuk-do (KR); Chang-Geun Lee, Daegu (KR); So-Young Choi, Uslan (KR); Sung-Ho Ryu, Gyeongsangbuk-do (KR); Youn-Dong Kim, Gyeongsangbuk-do (KR)

Correspondence Address: Tanya A Arenson;Medlen & Carroll, 101 Howard Street, Suite 350, San Francisco, CA, 94105, US

(21) Appl. No.: 10/543,999

(22) PCT Filed: Jan. 30, 2004
(86) PCT No.: PCT/KR04/00177

§ 371 Date: Aug. 24, 2005

(30) Foreign Application Priority Data
Jan. 30, 2003 (KR) 10-2003-0006393

(51) Int. Cl. A61K 48/00; A61K 39/21

(52) U.S. Cl. 424/188.1; 514/44

(57) ABSTRACT

Disclosed is a vaccine composition that includes a peptide adjuvant, and a DNA vaccine encoding an immunogenic protein. Also, the present invention discloses a method of enhancing immune responses, which is based on the administration of the vaccine composition.

### TECHNICAL FIELD

The present invention relates, in general, to a DNA vaccine composition, and more particularly, to a vaccine composition comprising a peptide adjuvant along with a DNA vaccine encoding an immunogenic protein. Also, the present invention is concerned with a method of enhancing immune responses, which is based on the administration of the vaccine composition.

### BACKGROUND ART

When a DNA vector carrying a gene that encodes a viral, bacterial, parasitic or tumor antigen is introduced into a host cell and expresses the antigen therein, such a naked DNA vector is called ‘polynucleotide vaccine (PNV)’ or ‘DNA vaccine’. Unlike protein- or peptide-based subunit vaccines, the DNA vaccines are capable of inducing antigen-specific humoral immune responses as well as cellular immune responses, in particular, strong Th1 and CTL-mediated immune responses. Also, the DNA vaccines are safe, easily manufactured and economical. Due to these potential advantages, the DNA vaccines have raised a lot of interest as alternatives for the conventional recombinant protein vaccines or recombinant viral vector vaccines. The conventional recombinant protein vaccines induce mainly humoral immune responses, and thus are not effective in inducing immune responses to intracellular pathogens, such as viruses.

Although the DNA vaccines have many benefits, the DNA vaccines are required to be improved for clinical application to humans. The DNA vaccines are known to induce lower immune responses than the recombinant viral vectors (Donnelly J J, Ulmer J B, Shiver J W and Liu M A. DNA vaccines. Annu Rev Immunol 1997, 15:617-48; Leitner W W, Ying H and Restifo N P. DNA and RNA-based vaccines: principles, progress and prospects. Vaccine 1999, 18:765-77). In addition, currently, there is no report for DNA vaccines capable of completely preventing a specific disease by their individual use. For this reason, a lot of effort was made to enhance the efficacy of the DNA vaccines (Rodriguez F and Whitton J L. Enhancing DNA immunization. Virology 2000, 268:233-8; Schneider J, Gilbert S C, Blanchard T J, et al. Enhanced immunogenicity for CD8+ T cell induction and complete protective efficacy of malaria DNA vaccination by boosting with modified vaccinia virus Ankara. Nat Med 1998, 4:397-402).

Various strategies to enhance the potency of the DNA vaccines may be classified into the following four categories.

The first approach is to increase the expression levels of the antigen in the target cells either by enhancing transcription efficiency from DNA or by enhancing transfection efficiency of the DNA vaccine into APC or muscle cells. Antigen expression can be elevated by use of a new promoter or genetic elements capable of increasing transcription of the antigen, or by optimization of codon usage (Rodriguez F and Whitton J L. Enhancing DNA immunization. Virology 2000, 268:233-8; Uchijima M, Yoshida A, Nagata T, Koide Y. Optimization of codon usage of plasmid DNA vaccine is required for the effective MHC class I-restricted T cell responses against an intracellular bacterium. J Immunol 1998, 161:5594-9; Deml L, Bojak A, Steck S et al. Multiple effects of codon usage optimization on expression and immunogenicity of DNA candidate vaccines encoding the human immunodeficiency virus type 1 Gag protein. J Virol 2001, 75:10991-1001). Various transfectants can be used for efficient transfection of the DNA vaccine into APC or muscle cells, or the probability for DNA to meet APC can be increased by directly inoculating the DNA vaccine into lymphoid organs (Rodriguez F and Whitton J L. Enhancing DNA immunization. Virology 2000, 268:233-8; Perrie Y, Frederik P M, Gregoriadis G. Liposome-mediated DNA vaccination: the effect of vesicle composition. Vaccine. 2001, 19:3301-10; Maloy K J, Erdmann I, Basch V et al. Liposome-mediated DNA vaccination: the effect of vesicle composition. Vaccine. 2001, 19:3301-10).

The second approach is to enhance immunogenicity of a target antigen through its modification. This approach involves increasing the intracellular degradation of the antigen, targeting the antigen into MHC molecules, and producing the antigen as a fusion protein with, for example, the non-toxic Fragment C (FrC) of tetanus toxin. Fusion of an antigen with the cytosolic region of LIMP-II (lysosomal integral membrane protein II) or ubiquitin facilitates the targeting of the antigen to the proteasome or lysosome for degradation, resulting in an increase in epitope presentation by MHC molecules (Rodriguez F and Whitton J L. Enhancing DNA immunization. Virology 2000, 268:233-8: Anderson R, Gao X M, Papakonstantinopoulou A, Fairweather N, Roberts M, Dougan G. Immunization of mice with DNA encoding fragment C of tetanus toxin. Vaccine 1997, 15:827-9; Zhu D, Rice J, Savelyeva N, Stevenson F K. DNA fusion vaccines against B-cell tumors. Trends Mol Med 2001, 7:566-72).

The third approach is to enhance the potency of the DNA vaccines by employing chemical compounds. Immune responses can be enhanced by inoculating the DNA vaccines in a mixture form with alum, vaxfectin or monophosphoryl lipid (MPL) A (Wang S, Liu X, Fisher K et al. Enhanced type I immune response to a hepatitis B DNA vaccine by formulation with calcium- or aluminum phosphate. Vaccine 2000, 18:1227-35; Hartikka J, Bozoukova V, Ferrari M et al. Vaxfectin enhances the humoral immune response to plasmid DNA-encoded antigens. Vaccine 2001; 19:1911-23; Lodmell D L, Ray N B, Ulrich J T, Ewalt L C. DNA vaccination of mice against rabies virus: effects of the route of vaccination and the adjuvant monophosphoryl lipid A (MPL). Vaccine 2000, 18:1059-66).

The fourth approach is to inoculate the DNA vaccines in combination with DNA expressing adjuvants, such as cytokines, chemokines or co-stimulatory molecules. This strategy can easily improve efficacy of the DNA vaccines (Scheerlinck J Y. Genetic adjuvants for DNA vaccines. Vaccine 2001; 19:2647-56). According to a recent report, when a genetically engineered mouse IL-12 (mIL-12 N220L) was co-immunized with a HCV E2 DNA vaccine, long-lasting CD8+ memory response was induced (Ha S J, Chang J, Song M K et al. Engineering N-glycosylation mutations in IL-12 enhances sustained cytotoxic T lymphocyte responses for DNA immunization. Nat Biotechnol 2002; 20:381-6).

Despite the aforementioned efforts, there is a need for the development of DNA vaccines capable of inducing more enhanced immune responses.

Thus, the present inventors attempted to enhance the immunogenicity of the aforementioned DNA vaccines, leading to the present invention. This research resulted in the finding that immune responses are enhanced when the conventional DNA vaccine was administered together with a peptide as an adjuvant, and more enhanced when an influenza nucleoprotein (NP) gene was administered in combination with the peptide adjuvant.

### DISCLOSURE OF THE INVENTION

The present invention relates to a vaccine composition comprising a peptide adjuvant and a DNA vaccine encoding an immunogenic protein.

The DNA vaccine is preferably a DNA plasmid. In addition, the DNA vaccine encodes an immunogen derived from a pathogenic organism selected from the group consisting of viruses, bacteria, parasites and fungi; preferably, an immunogen for induction of an immune response against a disease selected from among human immunodeficiency virus (HIV) infections, herpes simplex virus (HSV) infections, influenza virus infections, hepatitis A, hepatitis B, papillomavirus infections, tuberculosis, tumor growth, autoimmune diseases and allergies; and more preferably, an immunogen against an HIV.

The peptide adjuvant is a peptide composed of 2 to 30 amino acids, preferably, 4 to 10 amino acids, and more preferably, 6 amino acids. Most preferably, the peptide adjuvant is a Y peptide, WKYMV-d-M-NH₂, which contains a D-methionine in the sixth amino acid position instead of its L-isomer and a —NH₂ group in the C-terminus instead of the typical —COOH group.

The vaccine composition may further comprise a gene of an influenza virus. The influenza virus gene is preferably in the form of being inserted into a plasmid, and the influenza virus gene is preferably a neuraminidase-encoding gene.

In addition, the present invention relates to a method of enhancing immune responses by administering a vaccine composition comprising a peptide adjuvant and a DNA vaccine encoding an immunogenic protein.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows immune responses when a HIV DNA vaccine is administered in combination with a Y peptide; and

FIG. 2 shows immune responses when a HIV DNA vaccine is administered in combination with a Y peptide alone, an inflenza NP gene DNA alone, or both.

### BEST MODE FOR CARRYING OUT THE INVENTION

In an aspect, the present invention provides a vaccine composition comprising a peptide adjuvant and a DNA vaccine encoding an immunogenic protein.

As used herein, the term “immunogenic” refers to an ability to induce both humoral immune response and cellular immune response. The immunogenicity depends on the nature of an “immunogen” that is a substance capable of an immune response and the ability of an animal injected with the immunogen to respond to the immunogen. After contact with the immunogen, proliferation and maturation of T cells, NK cells, B cells, macrophages, etc. are induced, while cytokines such as interleukin-1 and interferon-γ are produced. Therefore, the immunogenicity of an immunogen can be identified by measuring the increased levels of cytokines after contact with the immunogen. Based on the degree of immune responses to an immunogen and the proportion of individuals showing immune responses, the immunogen may be concluded as a vaccine having excellent protective and therapeutic effects. In an embodiment of the present invention, the degree of the immune response is measured by an IFN-γ ELISPOT assay. However, the immune response may be measured by various application and utilization of assays. For example, humoral immune response may be measured by Single Radial Immunodiffusion Assay (SRID), Enzyme Immunoassay (EIA), Hemagglutination Inhibition Assay (HAI), and the like. Tests to measure cellular immune response include the determination of delayed-type hypersensitivity or the measurement of the proliferative response of lymphocytes to a target immunogen.

In the present specification, the DNA vaccine may be in the form of various recombinant vectors including a nucleotide sequence encoding one or more immunogens, for example, a plasmid vector, a cosmid vector, a bacteriophage vector and a viral vector, which is exemplified by an adenovirus vector, a retrovirus vector and an adeno-associated virus vector. Preferably, the DNA vaccine is in the plasmid vector form. As used herein, the term “vector” refers to a vehicle for introduction of a DNA fragment into a host cell. Such a vector includes regulatory elements necessary for gene expression of the coding sequence, including a promoter, an initiation codon, a stop codon and a polyadenylation signal. The vector may be prepared in various constructs according to intended use. The initiation and stop codons are generally considered to be a portion of a nucleotide sequence encoding an immunogenic target protein. Also, the initiation and stop codons are necessary to be functional in an individual to whom a genetic construct has been administered, and must be in frame with the coding sequence. Due to its typical disadvantage of low in vivo delivery efficiency, the DNA vaccine is necessary to be prepared using the most suitable promoter, an enhancer, etc., to increase expression of a target gene. Non-limiting examples of promoters useful for the expression of immunognes in eukaryotic cells include simian virus 40 (SV40) promoter, mouse mammary tumor virus (MMTV) promoter, human immunodeficiency virus (HIV) promoter such as the HIV Long Terminal Repeat (LTR) promoter, moloney virus promoter, cytomegalovirus (CMV) promoter such as the CMV immediate early promoter, epstein barr virus (EBV) promoter, rous sarcoma virus (RSV) promoter, as well as promoters from human genes such as β actin, human hemoglobin, human muscle creatine and human metalothionein. Non-limiting examples of the polyadenylation signals include SV40 poly(A) sequence and LTR poly(A) sequence. The enhancer may be selected from the group including, but not limited to, human actin, human myosin, human hemoglobin, human muscle creatine and viral enhancers such as those from CMV, RSV and EBV. In addition, other elements, such as a Kozak region, may also be contained in the genetic construct. In particular, an expression vector including CMV immediate early promoter, enhancer sequence, SV40 replication origin and poly(A) sequence is preferable. In the present invention, a pGX10 vector is used, which satisfies the aforementioned requirements. As used herein, the term “expression vector” refers to a genetic construct including essential regulatory elements operably linked to a coding sequence to allow immunogens to be expressed. Such a genetic construct, that is, the expression vector, may be prepared and purified by a standard recombinant DNA technique. The DNA vaccine may be used in the form of a naked DNA, being packaged in liposomes, for example, lecithin liposomes, or being coated onto colloidal gold particles.

The vector may include various genes encoding immunogens that act as antigens inducing immune responses. When two or more antigens are intended to be coded in a single vector, a single gene expression system, in which several structural genes are consecutively arranged under a single promoter and expressed in a fused hybrid form, or a multiple promoter system, may be employed. In the multiple promoter system, expression of each gene is regulated under a different promoter. Each antigenic polypeptide expressed may include a variant which contains one or more deletions, insertions, substitutions or other modifications and thus has a different amino acid sequence from the wild-type polypeptide. However, such a variant, like the wild-type polypeptide, should have an ability to stimulate humoral immune response or cellular immune response.

Preferably, the DNA vaccine contained in the vaccine composition of the present invention includes a nucleotide sequence that is operably linked to regulatory elements necessary for gene expression in eukaryotic cells and is in a DNA plasmid form. Such a DNA vaccine is preferably a plasmid that includes a gene encoding an immunogen derived from viruses, bacteria, parasites and fungi. The above immunogen may induce immune responses against, for example, human immunodeficiency virus (HIV) infections, herpes simplex virus (HSV) infections, influenza virus infections, hepatitis A, hepatitis B, papillomavirus infections, tuberculosis, tumor growth, autoimmune diseases and allergies.

As used herein, the term “adjuvant”, which is a substance that non-specifically stimulates the immune responses to an antigen at the early stage of activation of immune cells, refers to an agent, a molecule, etc., that is not an immunogen to a host but intensifies the immunity of the host by enhancing the activity of the immune cells. The adjuvant was reported to act by various mechanisms, for example, by increasing the surface area of an antigen, by prolonging the stay of an antigen in the body to allow the antigen enough time to approach to the lymphoid system, by targeting an antigen to macrophages, and by activating macrophages (H. S. Warren et al., Annu. Rev. Immunol., 4:369(1986)). The typical examples of the adjuvant include Freund's adjuvant, aluminum hydroxide gel, muramyl dipeptide and lipopolysaccharide (LPS). Such an adjuvant may be used alone or in combination with other pharmaceutical agents. In particular, in the present invention, the adjuvant means a peptide enhancing the immune response when used in combination with the DNA vaccine.

A peptide adjuvant used according to the present invention is a peptide composed of preferably 2 to 30 amino acids, more preferably, 3 to 15 amino acids, further preferably, 4 to 10 amino acids, still further preferably, 5 to 7 amino acids, and most preferably, 6 amino acids. Preferably, the adjuvant is a biodegradable material that can be removed from a tissue when immunoenhancing activity thereof is terminated. In an embodiment of the present invention, the peptide is a Y peptide (WKYMV-d-M-NH₂) that has the amino acid sequence of WKYMVM and contains a D-methionine in the sixth amino acid position instead of its L-isomer and a —NH₂ group in the C-terminus instead of the typical —COOH group.

The Y peptide used in the practice of the present invention is a hexapeptide. Hexapeptides having a certain sequence were known to activate immune cells. Among them, XKYX(P/V)M (X is any amino acid), in particular, WKYMVM-NH₂ or WKYMVm(WKYMV-d-M-NH₂) was identified as a potent activator of immune cells. This activity is mediated through the binding of the hexapeptide to pertussis toxin-sensitive G protein coupled receptor, leading to activation of PLC, increased intracellular Ca₂+ levels and finally phosphoinositide hydrolysis. Recently, the receptors with which the WKYMVM-NH₂ and WKYMVm(WKYMV-d-M-NH₂) peptides interact were reported to be FPR (formyl peptide receptor) and FPRL1 (formyl peptide receptor-like1), which play critical roles in inflammation and immune response, and to induce activation of various phagocytic responses, such as chemotaxis, superoxide generation and exocytosis, by stimulating the FPR and the FPRL1. As long as the peptide adjuvant, used in the vaccine composition of the present invention, enhances the immune response to a DNA vaccine when used in combination with the DNA vaccine, its amino acid sequence may include substitutions of some amino acid residues with structurally or functionally similar amino acids, additions of new amino acids or deletions of some amino acid residues.

In detail, the present inventors administered to mice a HIV DNA vaccine expressing HIV Gag and Env proteins, pGX10-GE, in combination with a synthetic Y peptide WKYMVm(WKYMV-d-M-NH₂), and conducted an IFN-γ ELISPOT assay using splenocytes isolated from the mice. In the ELISPOT assay, a Gag peptide pool (consisted of 20-amino-acid peptides that encompassed the entire HIV Gag sequence with 10-amino-acid overlaps) was used for Gag-specific stimulation, while a Balb/c mouse CD8⁺ T cell epitope (V3 peptide: RIQRGPGRAFVTIGK) was used for Env-specific stimulation. Immune responses were enhanced with the combinational use of the DNA vaccine with the Y peptide, and affected by administered amounts of the Y-peptide. In particular, the highest immune response was found when the Y peptide dosage was 2 μg. When the cells were stimulated with V3 peptide, the combinational administration of the DNA vaccine with 2 μg of the Y peptide induced about 3.8-fold higher response to Env than a control immunized with the pGX10-GE alone. However, the case of using 10 μg of the Y peptide was found to induce a slightly lower immune response than the case of using 2 μg of the Y peptide. This immunoenhancing effect of the Y peptide was further demonstrated by the cell stimulation with Gag peptide pool. When 30 μg of the pGX10-GE was administered alone, the Gag-specific INF-γ ELISPOT response (38 SFC/10⁶ splenocytes) was just slightly higher than a background level. Such a very weak immune response was elevated by about 3.8 times through the combinational administration of the DNA vaccine with 0.08 μg of the Y peptide, and by a maximum of 10 times through the combinational administration of the DNA vaccine with 2 μg of the Y peptide. Similar to the Env-specific response, upon the stimulation with Gag peptide pool, the case of using 10 μg of the Y peptide was found to induce a slightly lower immune response than the case of using 2 μg of the Y peptide.

The ELISPOT assay, used for evaluating the immunoenhancing activity of the vaccine composition of the present invention, is a highly-sensitive microplate-based assay for the detection of the relative number and frequencies of cytokine-secreting cells. In brief, cytokine-secreting cells are incubated in cytokine-specific capture antibody-coated wells, and washed while a secreted analyte (cytokine) captured by the immobilized antibody is not washed off. The captured analyte is incubated with an analyte-specific biotinylated detection antibody, and subsequently incubated with alkaline phosphatase-conjugated streptavidin. Then, a substrate is added to each well, and the formed spots are counted. The number of spots represents the frequency of antigen-specific lymphocytes secreting the analyte, that is, the cytokine. A commonly used cytokine is gamma interferon. In the present invention, gamma interferon was detected in the ELISPOT assay. The increased gamma interferon secretion indicates that the vaccine composition of the present invention, including the Y peptide as an adjuvant, effectively enhances Th1-type immune responses.

The vaccine composition of the present invention may further include a gene sequence of an influenza virus. Preferably, the influenza virus gene is an influenza nucleoprotein (NP) gene.

The influenza NP gene includes a gene that has a sequence homology of 70% or more, preferably, 80% or more, more preferably, 90% or more, and most preferably, 95% or more, with a gene encoding the influenza NP protein, and encodes a protein with the activity of the NP protein. The whole or a portion of such a gene may be used. When a portion of the influenza NP gene is intended to be used, a gene encoding an amino acid sequence including 50% or more of the N-terminus or the C-terminus of the influenza NP protein may be employed.

The above influenza NP gene is preferably in the form of being inserted into an expression vector, especially, a plasmid. Examples of the expression vector into which an influenza NP gene DNA is inserted include an expression vector having a promoter selected from among CMV promoter, RSV promoter, β-actin promoter and SV40 promoter, and a transcription termination sequence selected from a SV40 poly(A) signal and a BGH terminator. In an embodiment of the present invention, a pGX10-NP vector was used, which was constructed by preparing a pTV-NP by inserting an influenza NP gene into a pTV2 expression vector and by inserting into a pGX10 vector a region corresponding to nueraminidase (NA) obtained from the pTV-NP. In cases of immune responses to Env, the combinational use of the Y peptide and the pGX10-NP induced immune responses in similar levels within an effective range to the cases in which the Y peptide and the pGX10-NP were individually used. However, in cases of immune responses to Gag, the combinational use of the Y peptide and the pGX10-NP induced relatively enhanced immune responses in comparison with the cases in which the Y peptide and the pGX10-NP were individually used.

The influenza virus gene may be administered in combination with the DNA vaccine composition at various time intervals depending on several factors, for example, in a mixture with the DNA vaccine composition, simultaneously with the DNA vaccine composition, or before or after administration of the DNA vaccine composition. An influenza virus polypeptide expressed by the expression vector may include a variant which contains one or more deletions, insertions, substitutions or other modifications and thus has a different amino acid sequence from the wild-type polypeptide. However, such a variant, like the wild-type polypeptide, should have an ability to stimulate humoral immune response or cellular immune response.

The vaccine composition of the present invention, including the DNA vaccine and the peptide and optionally the influenza virus gene, induces an immune response against a variety of diseases including HIV infections, HSV infections, hepatitis A, hepatitis B, papillomavirus infections, tuberculosis, tumor growth, autoimmune diseases and allergies, and thus, may be formulated into a pharmaceutical form suitable for preventing or treating such diseases.

The vaccine composition may be combined with a pharmaceutically acceptable excipient. Examples of the excipient include water, saline, dextrose, glycerol, ethanol and mixtures thereof. Also, the vaccine composition may further include an auxiliary substance, which is exemplified by a humectant, an emulsifier and a pH-buffering agent. The vaccine composition may be administered in the form of liquid solutions, powders, aerosols, capsules, or enteric coating tablets or capsules or suppositories.

It is not intended that the present invention be limited to a particular mode of administration. A variety of modes of administration are contemplated, including intraperitoneally, intravenously, intramuscularly, subcutaneously, orally, topically, intramucosally, intranasally, intrapulmonarily and rectally. Since the peptide is digested upon oral administration, for oral administration, active ingredients of the vaccine composition should be coated or formulated for protection from degradation in the stomach. In addition, the pharmaceutical composition may be administered using a certain apparatus capable of transporting the active ingredients into a target cell.

A dosage of the vaccine composition may vary depending on pharmaceutical formulation types, administration routes, a patient's age, body weight and pathogenic states, currently available therapeutic methods and treatment frequency. And gene expression levels upon the administration of a DNA vaccine may vary depending on strength of regulatory sequences such as transcription promoters used in DNA constructs and immunogenicity of expressed gene products. The dosage may be easily determined by those skilled in the art. For example, in case of direct administration to muscle tissues, an immunologically or preventively effective amount ranges from about 1 μg to 5 mg, and preferably, about 10 μg to 2 mg.

The vaccine composition of the present invention may be administered alone or in combination with other therapeutic agents. Upon combinational administration, the vaccine composition may be administered sequentially or simultaneously with a conventional therapeutic agent.

The present invention will be explained in more detail with reference to the following examples in conjunction with the accompanying drawings. However, the following examples are provided only to illustrate the present invention, and the present invention is not limited to them.

### EXAMPLE 1

### Preparation of DNA Vaccines

Preparation of pGX10-GE

A pGX10-GE, which is a DNA vaccine expressing HIV Gag and Env proteins, was prepared, as follows. After a HIV DNA vaccine vector, pTX-GE (Lee Ah et al. Vaccine 1999, 17: 773-9), was digested with MluI and HpaI, the excised 7.5-kb fragment was ligated with a 2.2-kb fragment obtained by digesting with MluI and XbaI a pGX10 vector (deposited at an international depositary authority, Korean Collection for Type Cultures (KCTC) under an accession number of KCTC 10212BP on Mar. 29, 2002).

Preparation of pGX10-NP

First, a pTV-NP (deposited at KCTC under an accession number of KCTC 10193BP on Feb. 27, 2002) was digested with NotI and XbaI. The obtained 1.5-kb fragment corresponding to the neuramidase (NA) of an influenza virus (A/PR/8/34) was inserted into a NotI/XbaI-digested pGX10 vector, thus yielding a pGX10-NP (about 5.1 kb).

### EXAMPLE 2

### Preparation of a Peptide Adjuvant

A Y peptide, Trp-Lys-Tyr-Met-Val-D-Met-NH₂ (WKYMV-d-M-NH₂, WKYMVMm), was synthesized, which contained a D-methionine in the sixth amino acid position instead of its L-isomer and a ‘—NH₂’ group in the C-terminus instead of the typical ‘—COOH’. A V3 peptide (RIQRGPGRAFVTIGK, a HIV Env Balb/c mouse CD8⁺ T cell epitope) and a HIV Gag peptide pool, which will be used later as stimulators in INF-γ ELISPOT assays, were synthesized by the Peptron Company (Korea). The HIV Gag peptide pool was prepared by synthesizing 20-amino-acid peptides that encompassed the entire HIV Gag sequence with 10-amino-acid overlaps.

### EXAMPLE 3

### Evaluation of Immunoenhancing Activity of the Y Peptide on DNA Vaccine

Test 1

BALB/c mice used in this test were purchased from the SLC company (Shizuoka, Japan), and all tests using the mice were carried out in an animal breeding facility maintaining an specific pathogen free (SPF) condition (POSTECH Company, Korea).

Six to eight week-old mice were intramuscularly injected with a mixture of 30 μg of the pGX10-GE and the Y peptide of various concentrations (0, 0.08, 0.4, 2 and 10 μg) in 100 μl of PBS (phosphate buffered saline). After six weeks, the mice were boosted with an equal condition of the pGX10-GE and the Y peptide as before. Two weeks after the boosting, two mice per group were sacrificed. Splenocytes were isolated from the mice, and subjected to an IFN-γ ELISPOT assay.

The IFN-γ ELISPOT assay was carried out, as follows.

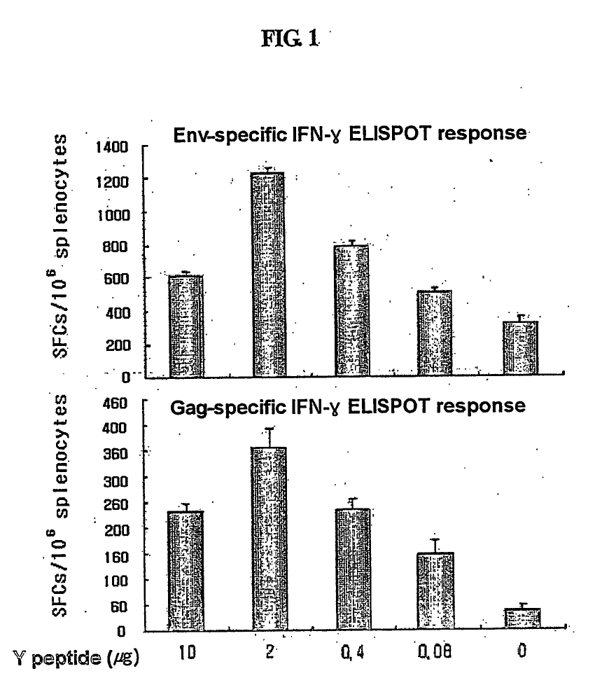

50 μl of a rat anti-mouse IFN-γ antibody (5 μg/ml, Pharmingen, Cat. No. 554431) was added to each well of a 96-well filtration plate (Millipore, Cat. No. MAIPN4550), followed by overnight incubation at 4° C. After removing the content of the plate, 200 μl of 10% fetal bovine serum (FBS)-containing DMEM (Dulbecco's Modified Eagle's Medium) was added to each well, and the plate was incubated at 37° C. for one hour. The splenocytes isolated from each group were diluted to 1-10×10⁷ cells/ml in a complete medium (RPMI-1640, 10% FBS, 2 mM L-glutamine, 50 μM β-mercaptoethanol, 100 unit penicillin/ml, 100 μg streptomycine/ml), and 100 μl (1-10×10⁶ cells/well) of the dilutant was added to each well. When the splenocytes were contained in a well in a density below 10⁶ cells, 50 μl of naïve splenocytes isolated from mice not immunized with the vaccine was added to the well up to 10⁶ cells. When a well contained 10⁶ splenocytes, the well was added with 50 μl of the complete medium. The V3 peptide for HIV Env-specific stimulation and the Gag peptide pool for HIV Gag-specific stimulation were individually added to each well after being mixed with 50 μl of the complete medium. The final concentration of each peptide was 1 μg/ml. The plate was incubated in a CO₂ incubator at 37° C. for 20 to 24 hrs. After removing the content from each well, each well was filled with water and incubated on ice for 15 min. After removing the water, each well was washed twice with PBST (0.1% Tween-20 in PBS). A biotinylated rat anti-mouse INF-γ antibody (Pharmingen, Cat. No. 554410) was diluted to 2 μg/ml with 2% BSA (in PBST), and 50 μl of the diluted antibody was added to each well. The plate was wrapped with kitchen wrap and allowed to stand at room temperature for 3 hrs. After removing the content from each well, each well was washed with PBST four times. Thereafter, 50 μl of a 1:2000 dilution of streptavidin-alkaline phosphatase (Pharmingen, Cat. No. 554065) in 2% BSA (in PBST) was added to each well. After the plate was wrapped with kitchen wrap and allowed to stand at room temperature for one hour, it was washed again with PBST eight times and developed with 50 μl/well of a BCIP/NBT solution (5-Bromo-4-chloro-3-indolyl-phosphate/nitroblue tetrazolium). When spots were formed at a suitable density, the reaction was stopped by washing the plate with water. After drying the plate, the formed spots were counted using an automatic ELISPOT reader. The number of INFγ-secreting cells was calculated by subtracting the value of the medium control not stimulated with the V3 peptide or the Gag peptide pool. The counted values were expressed as spot-forming cells (SFCs) per 10⁶ splenocytes. The value of each group was obtained using a mixture of splenocytes obtained from the two immunized mice. All experiments were performed in triplicate, and the data obtained from the INF-γ ELISPOT assay were presented as mean±SD. The results are given in Table 1 and FIG. 1, in which the error bars indicate SD values.

As shown in Table 1 and FIG. 1, the Y peptide enhanced the immunogenicity of the pGX10-GE. The case of using 2 μg of the Y peptide showed the highest effect on the enhancement of the immunogenicity of the pGX10-GE. The data shown in Table 1 were plotted in the graph of FIG. 1.

TABLE 1

| | Response to Env | Response to Gag |
|---|---|---|
| Y peptide (μg) | Mean ± SD | Mean ± SD |
| 10 | 617 ± 22 | 230 ± 19 |
| 2 | 1221 ± 38 | 355 ± 36 |
| 0.4 | 788 ± 25 | 233 ± 24 |
| 0.08 | 511 ± 19 | 146 ± 29 |
| 0 | 322 ± 36 | 38 ± 8 |

The immune response was enhanced when the pGX10-GE was administered in combination with the Y peptide, and affected by the dosage of the Y-peptide. In particular, the highest immune response was found when the Y peptide was used at a dose of 2 μg. In case of the Env-specific immune response, the combinational administration of the pGX10-GE together with 2 μg of the Y peptide induced an about 3.8-fold higher immune response than a control immunized with the pGX10-GE alone. However, the case of using 10 μg of the Y peptide was found to induce a slightly lower immune response than the case of using 2 μg of the Y peptide. This immunoenhancing effect of the Y peptide was further demonstrated by the cell stimulation with Gag peptide pool. When 30 μg of the pGX10-GE was administered alone, the Gag-specific INF-γ ELISPOT response (38 SFC/10⁶ splenocytes) was just slightly higher than the background level. Such a very weak immune response was elevated by about 3.8 times through the combinational administration of the pGX10-GE with 0.08 μg of the Y peptide, and by a maximum of 10 times through the combinational administration with 2 μg of the Y peptide. Similar to the Env-specific response, upon stimulation with Gag, the case of using 10 μg of the Y peptide was found to induce a slightly lower immune response than the case of using 2 μg of the Y peptide.

Test 2

The effect of the co-administration of the pGX10-NP with Y peptide on the immunogenicity of DNA vaccine was further investigated. Six to eight week-old mice were intramuscularly injected with vaccines listed in Table 2, below, in 100 μl of PBS.

TABLE 2

| Group | Vaccines |
|---|---|
| I | pGX10-GE (30 μg) + pGX10-mock (20 μg) |
| II | pGX10-GE (30 μg) + pGX10-mock (20 μg) + Y peptide |
| | (2 μg) |
| III | pGX10-GE (30 μg) + pGX10-mock (10 μg) + pGX10-NP |
| | (10 μg) |
| IV | pGX10-GE (30 μg) + pGX10-mock (10 μg) + pGX10-NP |
| | (10 μg) + Y peptide (2 μg) |

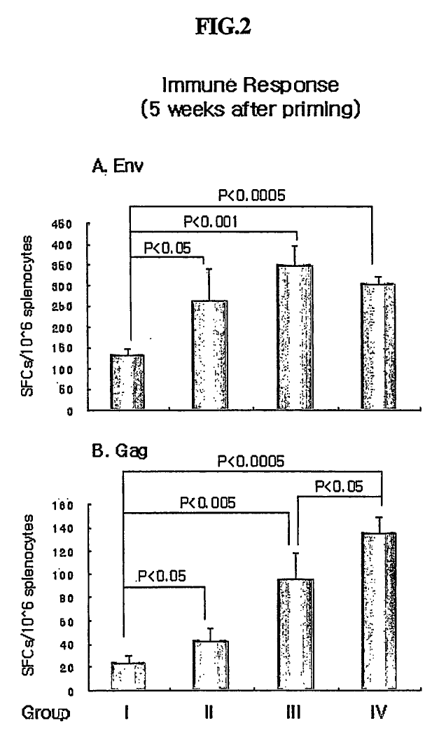

After five weeks, two mice per group were sacrificed. Splenocytes were isolated from the mice, and subjected to an IFN-γ ELISPOT assay. The results are given in FIG. 2 and Table 3. In the cases of immune responses to Env, the combinational use of the Y peptide and the pGX10-NP induced immune responses in similar levels within an effective range to the cases in which the Y peptide and the pGX10-NP were individually used. However, in the cases of immune responses to Gag, the combinational use of the Y peptide and the pGX10-NP induced relatively enhanced immune responses in comparison with the cases in which the Y peptide and the pGX10-NP were individually used.

TABLE 3

| | Response to Env | Response to Gag |
|---|---|---|
| Group | Mean ± SD | Mean ± SD |
| I | 133 ± 16 | 23 ± 6 |
| II | 263 ± 76 | 43 ± 11 |
| III | 348 ± 46 | 96 ± 22 |
| IV | 304 ± 17 | 135 ± 14 |

### INDUSTRIAL APPLICABILITY

As described hereinbefore, a DNA vaccine encoding an immunogenic protein can induce enhanced immune responses when used in combination with the peptide adjuvant or both the peptide adjuvant and the influenza NP DNA. Therefore, the vaccine composition according to the present invention can induce enhanced immune responses to various immunogens, and is useful for preventing or treating infectious diseases, autoimmune disease, cancer, etc.

[image]

[image]

1. A vaccine composition comprising:
(a) a peptide adjuvant; and
(b) a DNA vaccine encoding an immunogenic protein.

2. The vaccine composition as set forth in claim 1, wherein the DNA vaccine is a DNA plasmid.

3. The vaccine composition as set forth in claim 1, wherein the DNA vaccine encodes an immunogen derived from viruses, bacteria, parasites or fungi.

4. The vaccine composition as set forth in claim 3, wherein the DNA vaccine encodes an immunogen for induction of immune responses against a disease selected from among human immunodeficiency virus (HIV) infections, herpes simplex virus (HSV) infections, influenza virus infections, hepatitis A, hepatitis B, papillomavirus infections, tuberculosis, tumor growth, autoimmune diseases and allergies.

5. The vaccine composition as set forth in claim 4, wherein the DNA vaccine encodes an HIV immunogen.

6. The vaccine composition as set forth in claim 1, wherein the peptide adjuvant consists of 2 to 30 amino acids.

7. The vaccine composition as set forth in claim 6, wherein the peptide adjuvant is a Y peptide WKYMV-d-M-NH₂.

8. The vaccine composition as set forth in claim 1, further comprising a gene of an influenza virus.

9. The vaccine composition as set forth in claim 8, wherein the gene of the influenza virus is in the form of being inserted into a plasmid.

10. The vaccine composition as set forth in claim 9, wherein the gene of the influenza virus is a gene encoding neuraminidase.
